(19) United States
(12) Patent Application Publication
(10) Pub. No.: US 2025/0243172 A1
(43) Pub. Date: Jul. 31, 2025

(54) NLRP3 INFLAMMASOME INHIBITORS AND COMPOSITIONS AND USES THEREOF

(71) Applicant: VIRGINIA COMMONWEALTH UNIVERSITY, Richmond, VA (US)

(72) Inventors: Shijun ZHANG, Richmond, VA (US); Yiming XU, Richmond, VA (US)

(21) Appl. No.: 18/854,809

(22) PCT Filed: Apr. 4, 2023
(86) PCT No.: PCT/US2023/017360

§ 371 Date: Oct. 7, 2024

Related U.S. Application Data
(60) Provisional application No. 63/333,209, filed on Apr. 21, 2022.

(51) Int. Cl. C07D 249/06; A61K 31/4192; A61K 31/4245; A61K 31/4439; C07D 249/04; C07D 401/04; C07D 403/06; C07D 403/10; C07D 413/10

(52) U.S. Cl. CPC C07D 249/06; A61K 31/4192; A61K 31/4245; A61K 31/4439; C07D 249/04; C07D 401/04; C07D 403/06; C07D 403/10; C07D 413/10

(57) ABSTRACT

NLRP3 selective inhibitors (NSIs) as anti-inflammatory agents are provided, as are methods of using NSIs to inhibit inflammation and prevent or treat diseases and conditions associated with inflammation, such as Alzheimer's disease, Parkinson's disease, multiple sclerosis, traumatic brain injury, acute myocardial infarction, heart failure, arthritis, diabetes, gout, COVID-19, and autoinflammatory diseases.

### STATEMENT REGARDING FEDERALLY SPONSORED RESEARCH OR DEVELOPMENT

This invention was made with government support under Grant Number R01 AG058673 and U01AG076481 awarded by the National Institutes of Health (NIH). The government has certain rights in the invention.

### BACKGROUND OF THE INVENTION

### Field of the Invention

The invention generally relates to small molecule compounds that modulate the innate immune responses and methods of their use to inhibit pathological responses associated with innate immune dysregulation or over-activation. In particular, the invention provides small molecule NLRP3 selective inhibitors (NSIs), and methods of using the compounds and analogs thereof to prevent or treat NRLP3 inflammasome dysregulation associated diseases and conditions, such as multiple sclerosis (MS), Alzheimer's disease (AD), traumatic brain injury (TBI), Parkinson's disease (PD), acute myocardial infarction (AMI), heart failure, gout, rheumatoid arthritis, COVID-19, macular diabetes, degeneration, and autoimmune/autoinflammatory diseases.

### Background of the Invention

Inflammasomes are cytosolic multiprotein complexes that play key roles in the innate immune responses in recognition of pathogen- and damage-associated molecular patterns (PAMPs and DAMPs). The canonical activation of inflammasomes leads to caspase-1 activation and subsequent release of pro-inflammatory cytokines interleukin (IL)-1β and IL-18.

Consequently, an array of inflammatory responses and/or pyroptosis will be initiated. To date, three types of inflammasomes assembled by sensor proteins have been extensively studied. This includes the nucleotide-binding oligomerization domain [NOD] leucine rich repeat [LRR]-containing receptors (NLRs), protein absent in melanoma 2 (AIM2), and pyrin. Typically, inflammasomes are supramolecular assemblies composed of a sensor protein, an adaptor protein (apoptosis-associated speck-like protein containing a caspase recruitment domain—ASC), and an effector component, e.g., pro-caspase-1. Among them, the NLRP3 can recognize a plethora of signals, e.g., extracellular ATP, β-amyloid (Aβ), nigericin, and biologically relevant crystals including alum, calcium pyrophosphate dihydrate (CPPD), monosodium urate (MSU), silica, and asbestos, via yet understood mechanisms. Cryopyrin-associated periodic syndrome (CAPS), a dominantly inherited auto-inflammatory disease, provides evidence to support the translational potential of targeting the NLRP3 inflammasome by its connection with gain-of-function mutations in NLRP3. Aberrant NLRP3 inflammasome activity is also thought to contribute to the pathogenesis of other complex diseases, notably metabolic disorders, obesity, atherosclerosis and neurodegenerative disorders such as Alzheimer's disease (AD) and Parkinson's diseases (PD).

Alzheimer's disease (AD) is the most common type of dementia, and >5 million Americans and up to 36 million individuals worldwide are currently affected by AD. In addition, >$260 billion is spent annually in the US alone on AD treatment and care, significantly exacerbating problems with an already overextended health care system, and is projected to become a dominant health care expenditure over the next 3 decades. Unfortunately, existing treatments provide only temporary symptomatic relief. Pathologically, AD is uniquely characterized by the presence of extracellular senile plaques and intracellular neurofibrillary tangles, with β-amyloid (Aβ) and hyper-phosphorylated tau being the ingredients, respectively. Potential disease-modifying therapeutics for AD have and are being tested, and most approaches under active pursuit are targeting Aβ. Recently, two monoclonal antibodies targeting Aβ, aducanumab and lecanemab, have been approved by the FDA for the treatment of AD.

Among the indicated AD risk factors, neuroinflammation has been recognized as an essential player. Genetic, pathological, and epidemiological studies strongly support the essentiality of neuroinflammation in AD development and progression. Glial dysfunction due to inefficient phagocytosis or degeneration and elevated pro-inflammatory cytokines have been observed in preclinical AD models and in AD patients. Notably, no neuroinflammation was observed in the population with high Aβ plaque but without dementia, thus suggesting its causative roles in cognitive impairment. Studies also found that chronic inflammation can induce Aβ and tau pathologies, instead of just being a passive response activated by plaques and tangles. Recent studies also showed neuroinflammation decades before cognitive impairments. Collectively, the evidence strongly support strategies targeting neuroinflammation for the treatment of AD. Although epidemiological studies touted the benefits of non-steroid anti-inflammatory drugs (NSAIDs) in reducing the risk of AD, clinical studies found no efficacy of NSAIDs in improving cognitive functions in AD patients. Several explanations have been put forth to explain this discrepancy between epidemiological and clinical studies, e.g., treatment timing and duration, the specific drugs being evaluated, and trial design. A recent analysis of the ANDI dataset showed that one particular NSAID, diclofenac, is associated with reduced AD risk and slower cognition decline, but may have a cyclooxygenase independent mechanism. Thus, attenuation of neuroinflammation by novel mechanisms of action (MOAs) may still hold great promise to provide effective treatments.

Recently, emerging evidence has suggested a link between NLRP3 inflammasome and AD development. NLRP3 can sense a plethora of exogenous and endogenous molecules including Aβ and tau aggregates to activate the NLRP3 inflammasome. The levels of NLRP3, ASC, caspase-1, and down-stream effectors including IL-1β and IL-18 were found to be upregulated in AD mouse models and AD patients. Recently, increased NLRP3 inflammasome activity, evidenced by the active caspase-1 and ASC levels, was also found in frontotemporal dementia (FTD) patients and in the tau22 FTD mouse model. Co-localization of NLRP3, ASC, and caspase-1 was also seen in mouse AD models. Also, both IL-1β and IL-18 have essential roles in AD pathologies, e.g., synaptic plasticity, Aβ, and tau.

In addition to the pathogenic effects of the NLRP3 inflammasome underlying AD, pharmacological and genetic downregulation of this complex in preclinical AD models suggested translational potential in developing AD therapeutics. Knockout of NLRP3 or caspase-1 ameliorated Aβ pathology and improved spatial memory functions in transgenic APP/PS1 mice. Another study employing 5XFAD mice carrying the ASC^{+/-} genotype also supported this notion. Furthermore, recent studies in Tau22 mice demonstrated that knockout of NLRP3 or ASC reduced tau phosphorylation and aggregation. Also, deletion of NLRP3 in aged mice showed protective activity from aging-related cognitive decline, suggesting its central role in the inflammatory responses of normal aging. Recent studies have also indicated NLRP3 inflammasome dysregulation is a mechanism in connecting gut microbiota change to neuroinflammation. This is consistent with the observation of increased NLRP3 inflammasome activity of monocytes from AD patients, and is in line with early studies demonstrating infiltration of peripheral monocytes to the CNS in AD. Collectively, these results indicate essential and convergent roles of the NLRP3 inflammasome axis in AD development.

Multiple sclerosis (MS) is an immune mediated and neurodegenerative disorder characterized by neuroinflammation and demyelination. Currently there is no cure for MS and current medications mainly speed up recovery, reduced relapse rates, or manage symptoms. The immunopathology of MS is characterized by the infiltration of myelin-reactive T cells into the central nervous system (CNS) and induction of demyelination which disrupts the communication of the nervous system. Although the exact etiology and pathogenesis of MS remain unknown, emerging evidence supports a critical role for NLRP3 inflammasome and IL-1β in the pathogenesis of MS. Clinical studies showed that expression of caspase-1, IL-1β, and IL-18 was elevated in MS plaques and peripheral mononuclear cells of MS patients. Intriguingly, MS-like lesions were observed in a Muckle-Wells syndrome (MWS) patient who had a disease-susceptible mutation in the Nlrp3 gene. Absence of the inflammasome products caspase-1, IL-1β and IL-18 rendered mice resistant to experimental autoimmune encephalomyelitis (EAE), a mouse model that mimics human MS. Animal studies have shown that NLRP3 deficiency substantially delayed onset and reduced severity of EAE symptoms, decreased neuroinflammation, demyelination and oligodendrocyte loss progression. Recently, the effectiveness of IFN-β, a drug that has been used for more than 15 years as a first-line treatment for human MS, was found to depend on NLRP3 inflammasome, suggesting that IFN-β may therapeutically target the NLRP3 inflammasome-IL-1β axis in MS.

NLRP3 inflammasome also plays critical roles in the inflammatory responses to myocardial injury during AMI. In the early phases of AMI, the acute ischemic injury induces the expression of NLRP3 inflammasome components (priming), which concomitantly provides the stimuli leading to NLRP3 activation and formation of the macromolecular aggregate (trigger), leading to an active inflammasome. Caspase-1 is detected in the heart starting 3-6 hours after ischemia and its activity peaks between 24 and 72 hours, while low grade activation persists for weeks after the initial insult. Reperfusion, while it effectively reduces infarct size, does not prevent activation of the NLRP3 inflammasome and leads to further injury through caspase-1-dependent inflammatory cell death. To support this notion, studies demonstrated that mice with genetic deletion of NLRP3 or ASC exhibited smaller infarct size in experimental AMI model, and reduced tendency toward adverse remodeling and heart failure, consistent with a previously reported central role of caspase-1 in AMI. Transgenic mice expressing constitutively active caspase-1, on the other hand, developed adverse cardiac remodeling and heart failure.

The outbreak of the coronavirus disease 2019 (COVID-19), caused by the severe acute respiratory syndrome coronavirus 2 (SARS-CoV-2), has evolved into a global pandemic. Although the approval of COVID-19 vaccines have significantly protected the general populations, the emergence of viral variants continues casting tremendous challenges and burdens on our society. The majority of patients with COVID-19 exhibit mild-to-medium symptoms, however, 5-10% of COVID-19 patients become significantly ill suffering from excessive immune response dysregulation and high mortality.¹ One of the clinical signs of critically ill COVID-19 patients is the resulting complications of acute respiratory distress syndrome (ARDS) and acute lung injury (ALI), which leads to respiratory and multi-organ failures, and ultimately patient death.² In addition, ARDS by itself is a life-threatening condition of seriously ill patients associated many risk factors.³ Thus, effective treatments to mitigate ARDS/ALI are urgently needed.

Recent studies have emerged to suggest a critical role for the NLRP3 inflammasome in the observed cytokine storm and the development of ARDS/ALI in COVID-19. Intriguingly, early studies have revealed the essential role of the NLRP3 inflammasome in the development of ARDS/ALI. Recent studies using animal models and COVID-19 patient samples also demonstrated NLRP3 activation by SARS-CoV-2 via multiple mechanisms. More importantly, NLRP3 activation is observed in COVID-19 patients and is associated with lesions of the nervous systems/lungs and disease severity. Furthermore, NLRP3 inflammasome is overly activated in elderly individuals and age is one of the strongest predictors of COVID-19 mortality with 80% of COVID-19 deaths in the USA being in people of >65 years old. In addition, many major risk factors associated with COVID-19 contraction, e.g., diabetes and obesity, have been demonstrated to have a strong link with NLRP3 inflammasome dysregulation.

In view of the involvement of the NLRP3 inflammasome in these and other disease processes, there is still a need for development of novel small molecule inhibitors targeting the NLRP3 inflammasome pathway.

### SUMMARY

Embodiments of the disclosure provide alkyl 4-(1-(substituted benzyl)-triazol-4-yl)benzoate type of analogs as novel NLRP3 selective inhibitors (NSIs). These compounds, for example as depicted in generic Formula I, represent novel therapeutic agents for treatment of diseases such as AD, MS, AMI, PD, TBI, COVID-19, heart failure, arthritis, diabetes, macular degeneration, and gout.

One aspect of this invention provides a compound of Formula I:

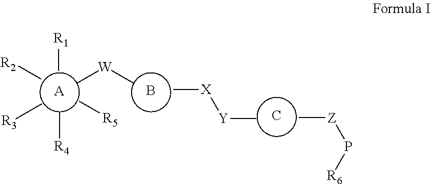

wherein
ring A is benzene, pyridine, pyrimidine, or 1,2-diazine;
R1 is unbranched, branched, saturated, unsaturated, cyclic or acyclic, substituted or unsubstituted C1-C8 alkyl or is unbranched, branched, saturated, unsaturated, cyclic or acyclic, substituted or unsubstituted C1-C8 alkoxyl, or is amino, nitro, OH, or halogen;
R4 is halogen, amino, nitro or cyano;
R2, R3, and R5 may be the same or different and are independently selected from H, C1-C8 unbranched, branched, saturated, unsaturated, cyclic or acyclic, substituted or unsubstituted alkyl, C1-C8 unbranched, branched, saturated, unsaturated, cyclic or acyclic, substituted or unsubstituted alkoxyl, C1-C8 unbranched, branched, saturated, unsaturated, cyclic or acyclic, substituted or unsubstituted alkylcarbonyl, halogen, hydroxyl, amino, nitro and cyano;
W is unbranched, branched, saturated, unsaturated, substituted or unsubstituted C1-C4 alkyl and may be present or absent;
ring B is 1,2,3-triazole, 1,2,4-triazole, 1,2-diazole, 1,3-diazole, pyrrole, 1,3-thiazole, or 1,3-oxazole;
X is unbranched, branched, saturated, unsaturated, substituted or unsubstituted C1-C4 alkyl and may be present or absent;
Y is O, NH, or S and may be present or absent;
ring C is benzene, pyridine, or another aromatic heterocycle;
Z is O, carbonyl, halogen, CF3, or C1-C8 unbranched, branched, saturated, unsaturated, cyclic or acyclic, substituted or unsubstituted alkyl;
P is O or S and may be present or absent; and
R6 is unbranched, branched, saturated, unsaturated, cyclic or acyclic, substituted or unsubstituted C1-C8 alkyl,
   or a pharmaceutically acceptable salt, solvate or hydrate thereof.

In one aspect, the compound is Formula II:

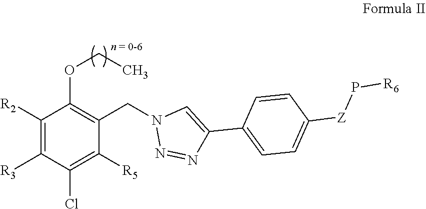

In one aspect, the compound is selected from:

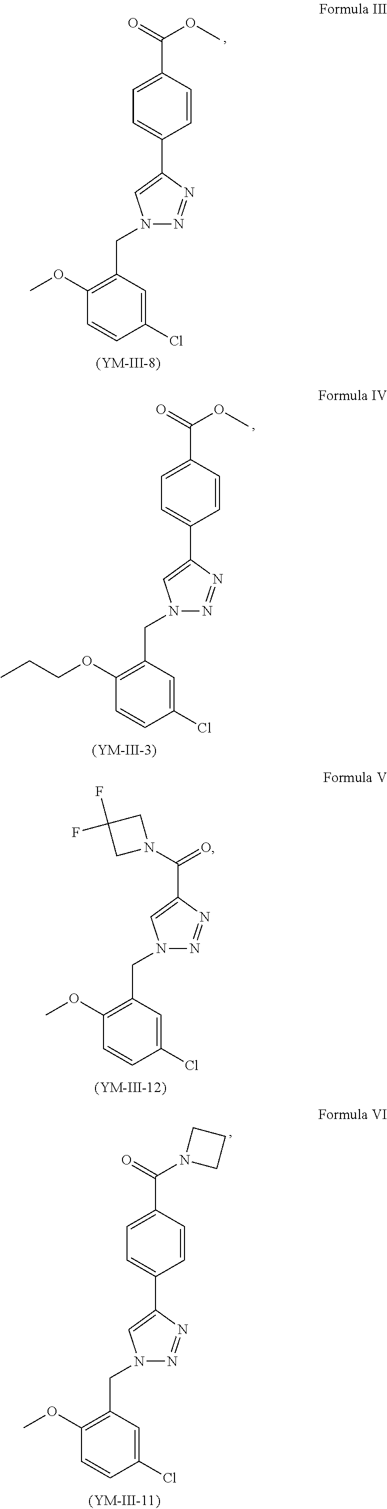

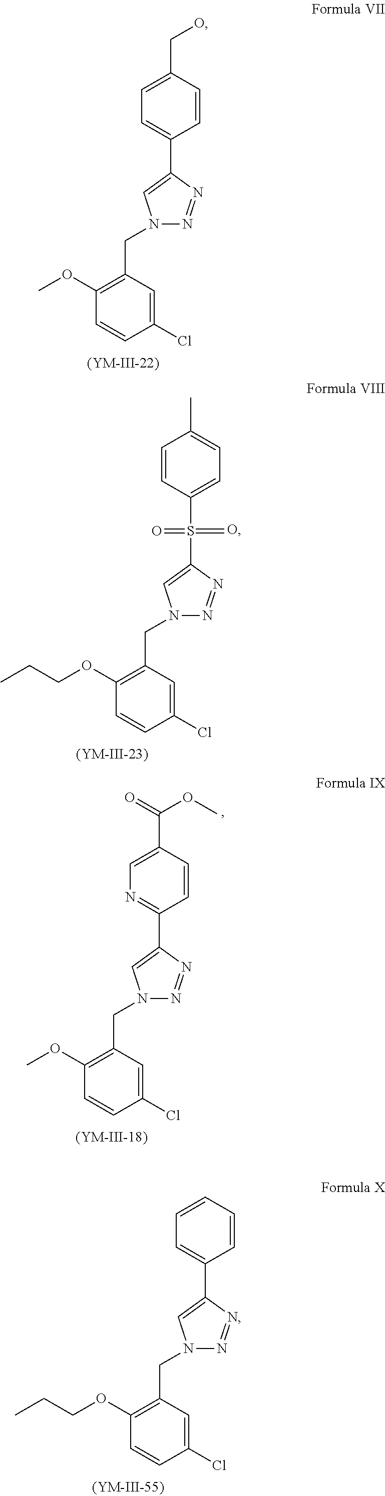

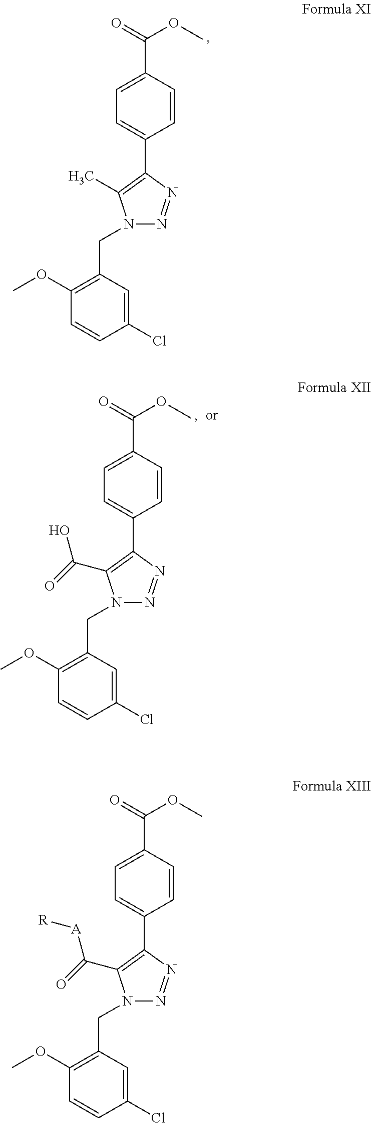

wherein R is unbranched, branched, saturated, unsaturated, cyclic or acyclic, substituted or unsubstituted C1-C8 alkyl and A is O or NH.

Another aspect of the invention provides a compound of Formula XIV:

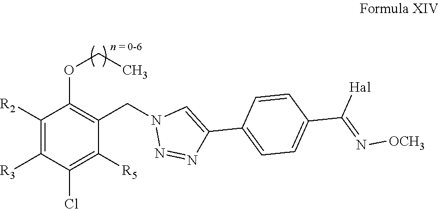

wherein
R2, R3, and R5 may be the same or different and are independently selected from H, C1-C8 unbranched, branched, saturated, unsaturated, cyclic or acyclic, substituted or unsubstituted alkyl, C1-C8 unbranched, branched, saturated, unsaturated, cyclic or acyclic, substituted or unsubstituted alkoxyl, C1-C8 unbranched, branched, saturated, unsaturated, cyclic or acyclic, substituted or unsubstituted alkylcarbonyl, halogen, hydroxyl, amino, nitro, and cyano; and
Hal is F or Cl,
   or a pharmaceutically acceptable salt, solvate or hydrate thereof.

Another aspect of the invention provides a compound of Formula XV and Formula XVI:

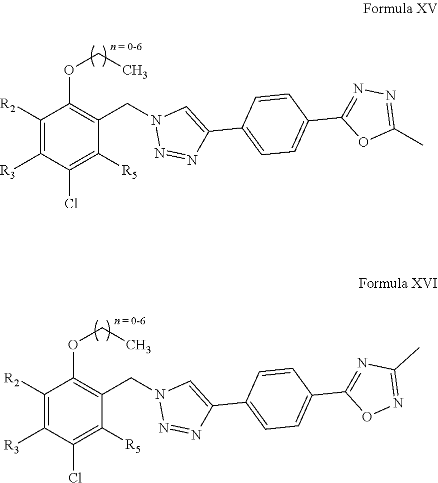

wherein
R2, R3, and R5 may be the same or different and are independently selected from H, C1-C8 unbranched, branched, saturated, unsaturated, cyclic or acyclic, substituted or unsubstituted alkyl, C1-C8 unbranched, branched, saturated, unsaturated, cyclic or acyclic, substituted or unsubstituted alkoxyl, C1-C8 unbranched, branched, saturated, unsaturated, cyclic or acyclic, substituted or unsubstituted alkylcarbonyl, halogen, hydroxyl, amino, nitro, and cyano.

The invention also provides compositions comprising each of these compounds, and variants thereof as described herein, combined with a physiologically acceptable carrier.

The invention also provides methods of preventing or treating NRLP3 inflammasome-associated disease, or inflammation associated with the disease, in a subject in need thereof, comprising a step of administering to said subject a therapeutically effective amount of a compound or composition as described herein.

In exemplary methods, the NRLP3 inflammasome-associated disease is selected from the group consisting of AD, MS, AMI, TBI, PD, heart failure, arthritis, diabetes, COVID-19, gout, macular degeneration, and an autoinflammatory condition.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

### BRIEF DESCRIPTION OF THE DRAWINGS

The accompanying drawings, which are incorporated in and constitute a part of this specification, illustrate embodiments of the invention and, together with a general description of the invention given above, and the detailed description given below, serve to explain the invention.

FIGS. 1A-R. Inhibition of the NLRP3 inflammasome by selected NSIs. IL-1β production by J774A.1 cells in response to LPS/ATP was measured by ELISA in the presence of selected NSIs at indicated concentrations. Error bar represents SEM.

FIGS. 2A-D. Inhibition of the NLRC4 and AIM2 inflammasomes by selected NSIs. IL-1β production by J774A.1 cells in response to LPS/poly(dA;dT) or LPS/Flagellin was measured by ELISA in the presence of selected NSIs at indicated concentrations. Error bar represents SEM.

### DETAILED DESCRIPTION

The following descriptions and examples illustrate some exemplary embodiments of the disclosed invention in detail. Those of the skill in the art will recognize that there are numerous variations and modifications of this invention that are encompassed by its scope. Accordingly, the description of a certain exemplary embodiment should not be deemed to limit the scope the present invention.

As described herein, the invention provides NSIs that inhibit the NLRP3 inflammasome activity, as are methods of their use to treat various NLRP3-inflammasome related diseases and conditions. The NSIs have the generic structures of Formula I:

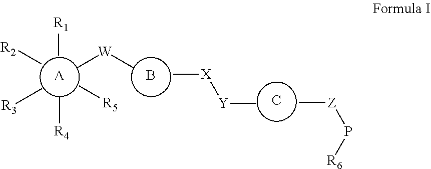

wherein
ring A is benzene, pyridine, pyrimidine, or 1,2-diazine;
R1 is unbranched, branched, saturated, unsaturated, cyclic or acyclic, substituted or unsubstituted C1-C8 alkyl or is unbranched, branched, saturated, unsaturated, cyclic or acyclic, substituted or unsubstituted C1-C8 alkoxyl, or is amino, nitro, OH, or halogen;
R4 is halogen, amino, nitro or cyano;
R2, R3, and R5 may be the same or different and are independently selected from H, C1-C8 unbranched, branched, saturated, unsaturated, cyclic or acyclic, substituted or unsubstituted alkyl, C1-C8 unbranched, branched, saturated, unsaturated, cyclic or acyclic, substituted or unsubstituted alkoxyl, C1-C8 unbranched, branched, saturated, unsaturated, cyclic or acyclic, substituted or unsubstituted alkylcarbonyl, halogen, hydroxyl, amino, nitro and cyano;
W is unbranched, branched, saturated, unsaturated, substituted or unsubstituted C1-C4 alkyl and may be present or absent;
ring B is 1,2,3-triazole, 1,2,4-triazole, 1,2-diazole, 1,3-diazole, pyrrole, 1,3-thiazole, or 1,3-oxazole;
X is unbranched, branched, saturated, unsaturated, substituted or unsubstituted C1-C4 alkyl and may be present or absent;
Y is O, NH, or S and may be present or absent;
ring C is benzene, pyridine, or another aromatic heterocycle;
Z is O, carbonyl, halogen, CF3, or C1-C8 unbranched, branched, saturated, unsaturated, cyclic or acyclic, substituted or unsubstituted alkyl;
P is O or S and may be present or absent; and
R6 is unbranched, branched, saturated, unsaturated, cyclic or acyclic, substituted or unsubstituted C1-C8 alkyl,
   or a pharmaceutically acceptable salt, solvate or hydrate thereof.

In one aspect, the compound is Formula II:

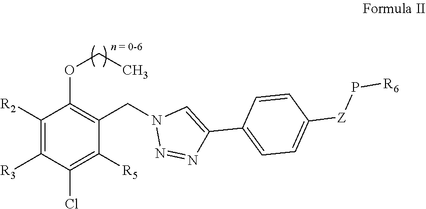

As used herein, any “R” group(s) such as, without limitation, R1, R2, R3, R4, R5, R6 and so on represent substituents that can be attached to the indicated atom. An R group may be substituted or unsubstituted. If two “R” groups are described as being “taken together” the R groups and the atoms they are attached to can form a cycloalkyl, aryl, heteroaryl, or heterocycle. R groups at different locations may be the same or different.

As used herein, any “W”, “X”, “Y”, “Z” or “P” group(s) represent substituents that can be attached to the indicated atom. Such group may be substituted or unsubstituted and such groups at different locations may be the same or different.

As used herein, “alkyl” refers to a straight or branched hydrocarbon chain that includes a fully saturated (no double or triple bonds) hydrocarbon group. The alkyl group may have 1 to 10 carbon atoms (whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated). The alkyl group may also be a medium size alkyl having 1 to 10 carbon atoms. Typical alkyl groups include, but are in no way limited to, methyl, ethyl, propyl, isopropyl, butyl, isobutyl, tertiary butyl, pentyl and hexyl, e.g. CH₃—, CH₃CH₂—, CH₃(CH₂)₂—, CH₃(CH₂)₃—, CH₃(CH₂)₄—, CH₃(CH₂)₅—, CH₃(CH₂)₆—, CH₃(CH₂)₇—, which may be substituted or unsubstituted, or propargyl.

As used herein, “alkylcarbonyl” refers to carbonyl attached to the above alkyl.

As used herein, “alkoxy” refers to the formula —OR wherein R is an alkyl, an alkenyl, an alkynyl, a cycloalkyl, a cycloalkenyl or a cycloalkynyl as defined as above. Exemplary alkoxyl groups include but are not limited to methoxy, ethoxy, n-propoxy, 1-methylethoxy (isopropoxy), n-butoxy, iso-butoxy, sec-butoxy and tert-butoxy, e.g. CH₃O—, CH₃CH₂O—, CH₃(CH₂)₂O—, CH₃(CH₂)₃O—, CH₃(CH₂)₄O—, CH₃(CH₂)₅O—, CH₃(CH₂)₆O—, CH₃(CH₂)₇O—, which may be substituted or unsubstituted, or ethynyloxy.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” is a heterocyclyl group derived from a heteroarene by removal of a hydrogen atom from any ring atom. Examples of heteroaryls include pyrrolidine, piperidine and pyridine. Examples of aromatic heterocycles include, but are not limited to, pyridine, furan, pyrrole, thiophene, indole, benzofuran, carbazole, quinoline, isoquinoline, imidazole, oxazole, pyrazole, pyridazine, pyrimidine, purine, etc.

The term “halogen atom” or “halogen” as used herein, means any one of the radio-stable atoms of column 7 of the Periodic Table of the Elements, such as, fluorine (F), chlorine (Cl), bromine (Br), and iodine (I).

“Substituted” refers to the inclusion of alkyl or a heteroatom or heteroatoms such as S, N, O, NO, OH, etc., within or attached to an alkyl chain or cyclic hydrocarbon.

An exemplary compound is depicted in Formulas III-XIII:

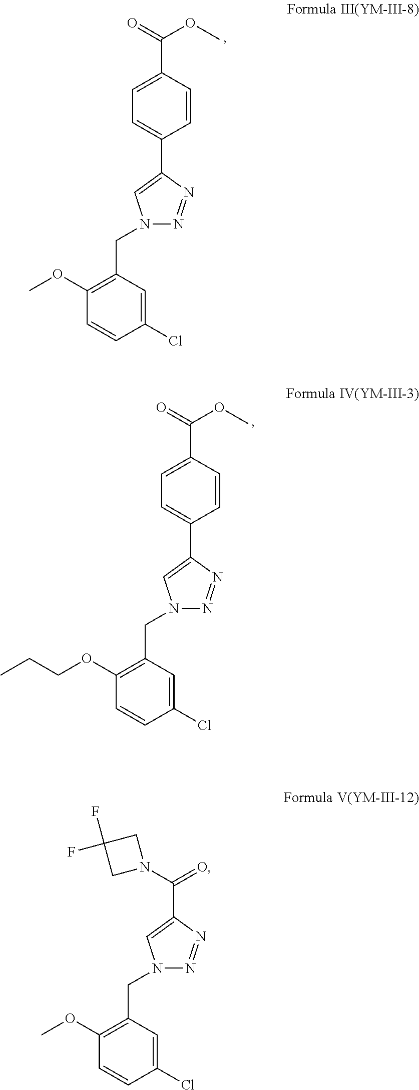

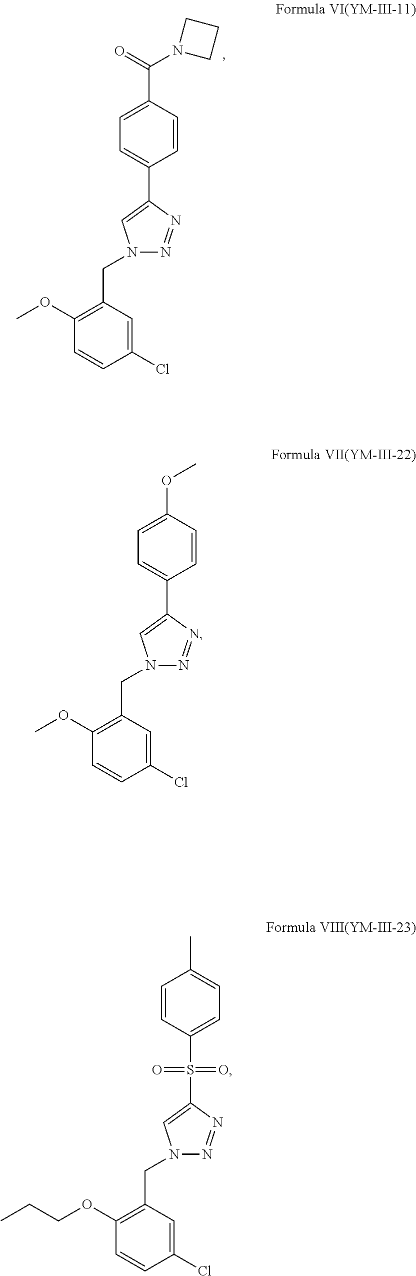

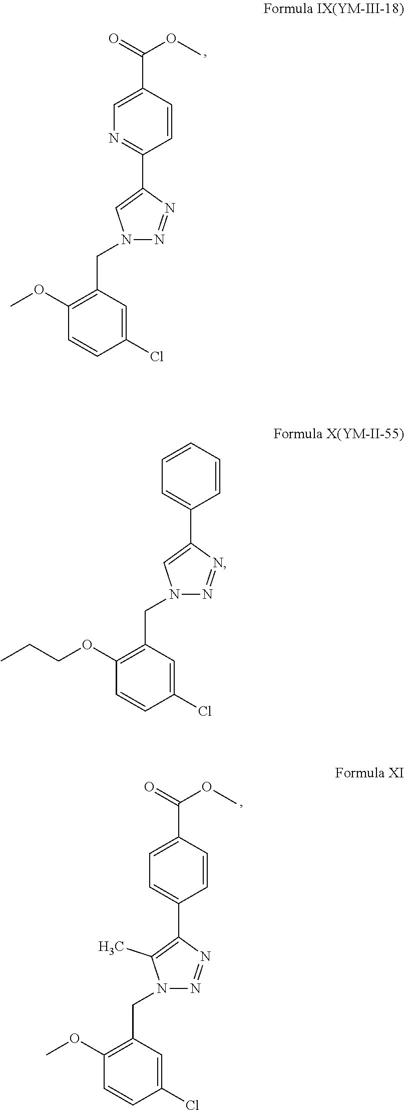

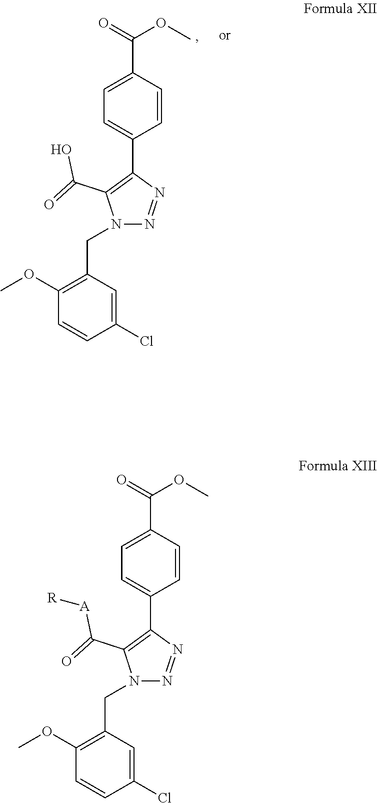

wherein R is unbranched, branched, saturated, unsaturated, cyclic or acyclic, substituted or unsubstituted C1-C8 alkyl and A is O or NH.

As used herein, the small molecule compound of YM-III-8 is Formula III, and the names may be used interchangeably.

As used herein, the small molecule compound of YM-III-3 is Formula IV, and the names may be used interchangeably.

As used herein, the small molecule compound of YM-III-12 is Formula V, and the names may be used interchangeably.

As used herein, the small molecule compound of YM-III-11 is Formula VI, and the names may be used interchangeably.

As used herein, the small molecule compound of YM-III-22 is Formula VII, and the names may be used interchangeably.

As used herein, the small molecule compound of YM-III-23 is Formula VIII, and the names may be used interchangeably.

As used herein, the small molecule compound of YM-III-18 is Formula IX, and the names may be used interchangeably.

As used herein, the small molecule compound of YM-II-55 is Formula X, and the names may be used interchangeably.

In some aspects, an exemplary compound is one of formula XVI-XVI:

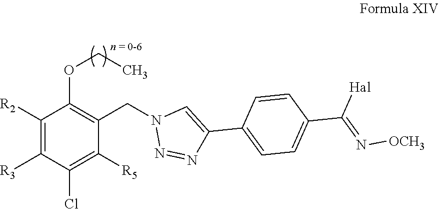

wherein
R2, R3, and R5 may be the same or different and are independently selected from H, C1-C8 unbranched, branched, saturated, unsaturated, cyclic or acyclic, substituted or unsubstituted alkyl, C1-C8 unbranched, branched, saturated, unsaturated, cyclic or acyclic, substituted or unsubstituted alkoxyl, C1-C8 unbranched, branched, saturated, unsaturated, cyclic or acyclic, substituted or unsubstituted alkylcarbonyl, halogen, hydroxyl, amino, nitro, and cyano; and
Hal is F or Cl.

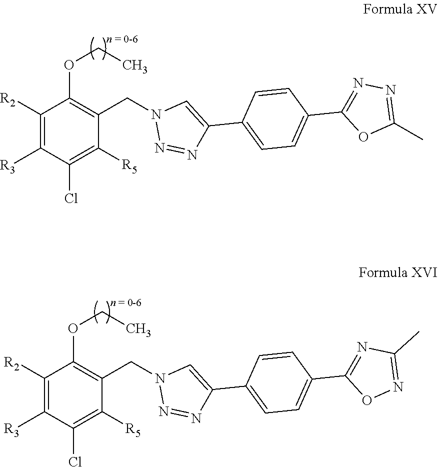

wherein
R2, R3, and R5 may be the same or different and are independently selected from H, C1-C8 unbranched, branched, saturated, unsaturated, cyclic or acyclic, substituted or unsubstituted alkyl, C1-C8 unbranched, branched, saturated, unsaturated, cyclic or acyclic, substituted or unsubstituted alkoxyl, C1-C8 unbranched, branched, saturated, unsaturated, cyclic or acyclic, substituted or unsubstituted alkylcarbonyl, halogen, hydroxyl, amino, nitro, and cyano.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvate forms with pharmaceutically acceptable solvents such as water, ethanol, or the like. In other embodiments, the compounds described herein exist in unsolvated form. Solvates contain either stoichiometric or non-stoichiometric amounts of a solvent and may be formed during the process of crystallization with a pharmaceutically acceptable solvent such as water, ethanol, or the like. Hydrates are formed when the solvent is water, or alcoholates are formed when the solvent is alcohol. In addition, a compound provided herein can exist in unsolvated as well as solvated forms. In general, the solvated forms are considered equivalent to the unsolvated form for the purpose of the compounds and methods provided herein.

Embodiments of the present invention provide compositions comprising the compounds described herein, and/or pharmaceutically acceptable salts of the compounds. The compositions are generally for use in preventing or treating inflammation, e.g. inflammation caused by formation and activity of NLRP3 inflammasomes, e.g. as associated with one or more diseases/conditions as described herein. The compositions include one or more substantially purified compounds as described herein, and a pharmacologically suitable (compatible) carrier. The preparation of such compositions is known to those of skill in the art. Typically, such compositions are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The active ingredients may be mixed with excipients which are pharmaceutically acceptable and compatible with the active ingredients.

Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. In addition, the compositions may contain other agents with different but complementary activities, e.g. other anti-inflammatory agents, analgesics, blood thinners, antihistamines, etc. If it is desired to administer an oral form of the compositions, various thickeners, flavorings, diluents, emulsifiers, dispersing aids or binders and the like may be added. The compositions of the present invention may contain any such additional ingredients so as to provide the composition in a form suitable for administration. The final amount of compound in the formulations may vary. However, in general, the amount in the formulations will be from about 1-99%. Still other suitable formulations for use in the present invention can be found, for example in Remington's Pharmaceutical Sciences, Philadelphia, Pa., 19th ed. (1995).

As used herein, “pharmaceutically acceptable salts” refers to the relatively non-toxic, inorganic and organic acid addition salts, and base addition salts, of compounds of the present invention. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-.beta.-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,” J. Pharm. Sci., 66, 1-19 (1977) which is incorporated herein by reference. Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use, ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

Precursors (generally inactive precursors) of the compounds which are metabolized after administration to yield the compounds/active agents described herein in an active form are also encompassed.

The therapeutic agents described herein are used alone or in combination with other suitable agents, e.g. other agents that prevent or treat inflammation (for example, by another mechanism), including but not limited to: IL-1R antagonists such as anakinra; monoclonal antibodies against interleukin 1β such as canakinumab (Ilaris); various interleukin 1 binding proteins such as rilonacept; and the like. Accordingly, the compositions provided herein may include one or more of these additional agents.

Method of use for small molecule compounds that inhibit the NLRP3 inflammasome activity are provided, as are methods to treat various NLRP3-inflammasome related diseases and conditions. Examples of the invention demonstrate the direct binding interactions of the inhibitors of the invention with the NLRP3 protein, thus interfering with formation of the NLRP3 inflammasome.

Embodiments of the invention provide a method of inhibiting, preventing or treating an NRLP3 inflammasome-associated inflammation or disease in a subject in need thereof, comprising a step of administering to said subject a therapeutically effective amount of a small molecule compound as described herein, or a pharmaceutical composition comprising the small molecule compound.

The NSIs disclosed herein are used to treat any disorder or condition associated with (e.g. caused by or related to or which exacerbates) unwanted NLRP3 inflammasome activation and/or consequences of such activation, e.g. unwanted production of pro-inflammatory cytokines pro-IL-1β and pro-IL-18. Such diseases/conditions may be caused by so-called sterile inflammation (e.g. various inflammatory diseases, second wave inflammation after heart attack, stroke or other ischemic or traumatic injury), or by inflammation that is caused by an infection (e.g. by an infectious organism such as a bacterium or virus). Such diseases and conditions result from a wide array of stimuli. For example, numerous microbes including various bacteria, viruses, fungi, and protozoan parasites can activate the NLRP3 inflammasome, e.g., the bacterial toxin nigericin has also been reported to induce the activation of NLRP3 by causing potassium efflux in a pannexin-1-dependent manner. In addition to microbial activators, endogenous “danger” signals such as ATP, monosodium urate (MSU) activate the NLRP3 inflammasome, as do various other types of cellular damage resulting e.g. from metabolic stress, ischemia and trauma. The NLRP3 inflammasome is implicated in metabolic disorders and sterile inflammatory responses including multiple sclerosis, arthritis, type II diabetes mellitus, gout and ischemia. A number of endogenous and exogenous crystalline molecules activate the NLRP3 inflammasome, e.g. uric acid crystals and calcium pyrophosphate dihydrate, the causative agents of gout and pseudogout respectively. Silica and asbestos particles, which cause the fibrotic lung disorders silicosis and asbestosis respectively, also activate the NLRP3 inflammasome.

Release of ATP from necrotic cells is a danger signal that activates the innate or sterile inflammatory immune response. Inhibiting NLRP3 inflammasome activation has beneficial effects in preventing the damage mediated by the sterile inflammatory response in diseases such as renal-, cardiac-, and cerebral-ischemia. In addition, necrosis-induced sterile inflammation in trauma and secondary to infections and sepsis are modulated by the inhibitors of the NLRP3 pathway described herein. The NLRP3 inflammasome can also be activated by molecules associated with stress or danger, including crystalline and particulate substances.

Examples of particular auto-inflammatory diseases which may be prevented or treated by the agents described herein include but are not limited to:
i) Joint, bone and muscle diseases such as rheumatoid arthritis, psoriatic arthritis, osteoarthritis, ankylosing spondylitis, erosive osteoarthritis of the hand, recurrent multifocal osteomyelitis, traumatic knee injury; relapsing polychondritis, etc;
ii) Hereditary systemic autoinflammatory diseases such as familial Mediterranean fever (FMF), cryopyrin-associated periodic syndrome (CAPS); Muckle-Wells Syndrome, TNF receptor-associated periodic syndrome (TRAPS), hyper-IgD syndrome (HIDS), periodic fever, aphthous stomatitis, pharyngitis and adenitis (PFAPA), deficiency of interleukin-1 (IL-1) receptor antagonist (DIRA), etc;
iii) Systemic inflammatory diseases such as systemic juvenile idiopathic arthritis, adult-onset Still's disease, Schnitzler syndrome, Behçet's disease, PFAPA (Periodic Fever, Apthous Sstomatitis, Pharyngitis, Adenitis), SAPHO (synovitis, acne, pustulosis, hyperostosis, osteitis) syndrome, macrophage activation syndrome, etc; and
iv) Common inflammatory diseases such as gout, Type 1 diabetes, Type 2 diabetes, metabolic syndrome, insulin resistance, stroke, heart attack, myocarditis, cardiac toxicity due to drug or radiation, ischemic heart disease, cardiomyopathy on a familial or genetic basis, heart failure, cardiac arrest and anoxic brain injury, acute and chronic lung injury due to infection, ischemia, toxin, trauma; dry eye syndrome, pustular psoriasis; neutrophilic dermatoses; acute or chronic hepatitis due a virus, toxin, ischemia or drug; acute or chronic renal injury due to ischemia, hypertension, diabetes, toxin or drugs; sepsis, septic shock; etc.

In one aspect, the compounds are used to treat Multiple sclerosis (MS). MS refers to all types of MS including relapse-remitting, secondary progressive, and primary progressive MS.

In one aspect, the compounds are used to treat neurodegenerative disorders such as AD, PD, ALS, and Huntington's disease.

In one aspect, the compounds are used to treat ARDS/ALI and cytokine storm associated with COVID-19.

The compositions (preparations) of the present disclosure may be administered by any of the many suitable means which are well known to those of skill in the art, including but not limited to: by injection (e.g. intravenous, intraperitoneal, intramuscular, subcutaneous, intra-aural, intraarticular, intramammary, and the like), by absorption through epithelial or mucocutaneous linings (e.g., nasal, oral, vaginal, rectal, gastrointestinal mucosal linings, and the like), by inhalation, orally, intranasally, by ingestion of a food or probiotic product containing the antimicrobial peptide, topically (e.g. on areas such as eyes, skin, in ears or on inflamed areas), as eye drops, via sprays, incorporated into dressings or bandages (e.g. lyophilized forms may be included directly in the dressing), etc. Generally, the mode of administration is by injection so as to effect systemic distribution of the agent, or locally by direct application, via an appropriate means, at or near a site of inflammation or a site where inflammation is likely to occur.

The amount of a compound that is administered varies depending on several factors, including the disease or condition being treated, the stage of the disease, the overall health of the subject, the subject's age, gender and weight, etc. In general, the amount is in the range of from about 0.01 to about 100 mg/kg of body weight, and usually is in the range of from about 1 to about 20 mg/kg of body weight. The subjects (patients) that are treated as described herein are generally mammals, e.g. humans, but veterinary applications of this technology are also encompassed, e.g. for companion pets such as cats and dogs.

The compounds of the disclosure are utilized to prevent and/or to treat conditions and/or diseases associated with (e.g. caused by) NRLP3 inflammasome activity (i.e. to treat NRLP3 inflammasome-associated inflammation). By “prevent” we mean that the compounds are administered prophylactically to a subject who is likely to develop the disease or condition, but before symptoms or indications of disease develop, or early in development. For example, subjects who have experienced AMI may be treated as described herein in order to prevent subsequent adverse cardiac remodeling during the “second wave” of inflammation. Alternatively, or in addition, the compounds may be administered in order to treat conditions/diseases that have already developed (e.g. when symptoms are already being exhibited, or are observable or measurable). In this case, administration of the compounds ameliorates and may reverse the symptoms, or at least arrest the disease (e.g. prevent further disease development or progress). Those of skill in the art will recognize that while a goal of prevention or treatment may be to completely prevent or alleviate disease symptoms, much benefit can also accrue if symptoms are not fully eradicated but are lessened, decreased or their onset is slowed, even though a full-blown cure is not effected.

Methods of treating NRLP3 inflammasome-related diseases are provided. Such methods may include a step of identifying a subject in need of such treatment (e.g. a subject with one or more symptoms of an NRLP3 inflammasome-related disorder, or a subject who is likely to develop such a disorder). For example, patients who have had MS may be treated, as patients for whom there is reason to suspect the relapse of MS is likely to occur.

The same is true for other conditions that are treated by the agents disclosed herein, i.e. a subject suitable for undergoing treatment may have one or more readily observable symptoms, or early symptoms, or a predisposition to development of the disease (e.g. genetically, due to life style, due to exposure to a substance that is known to cause inflammation, etc.) that is being treated.

As indicated above, the present invention inter alia provides the specified compounds for use in a method of: method of preventing or treating NRLP3 inflammasome-associated inflammation, including neuroinflammations associated with MS, AD, etc, as well as acute inflammation, or acute inflammatory response, which may occur in variety of illnesses in which an injury induces inflammation. Further, the present invention may provide the specified compound as an active therapeutic ingredient in the specified method. Further, the present invention may provide the specified compound for use in a method of treatment of the human or animal body by therapy, the method comprising the specified method.

Before exemplary embodiments of the present invention are described in greater detail, it is to be understood that this invention is not limited to any particular embodiments described herein and may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both limits, ranges excluding either or both of those included limits are also included in the invention. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Example

### Synthesis

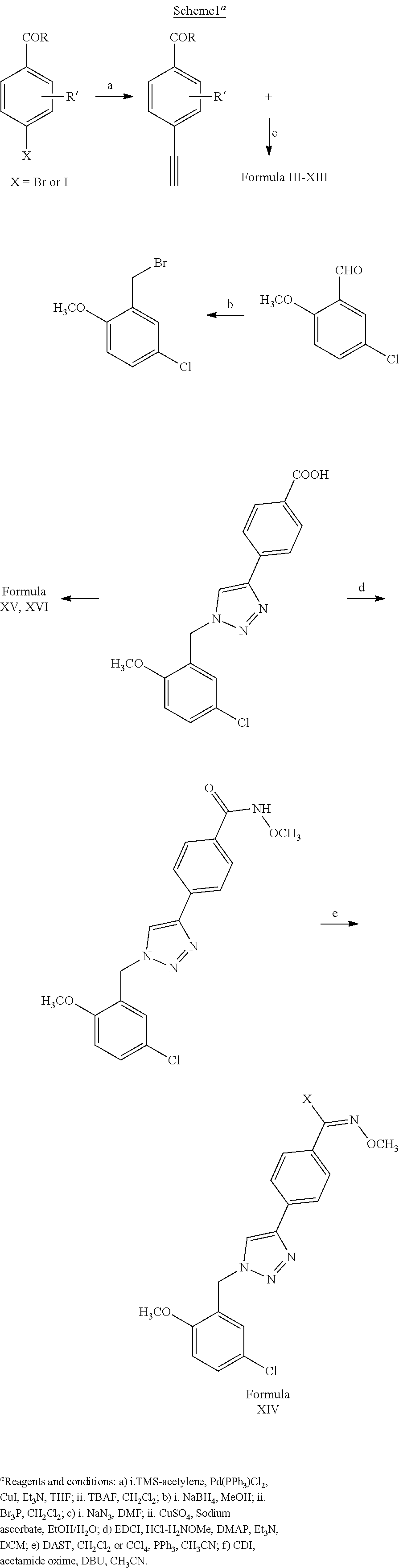

### NSIs Prevent the Release of IL-1β In Vitro and is Selective to NLRP3 Inflammasome Pathway

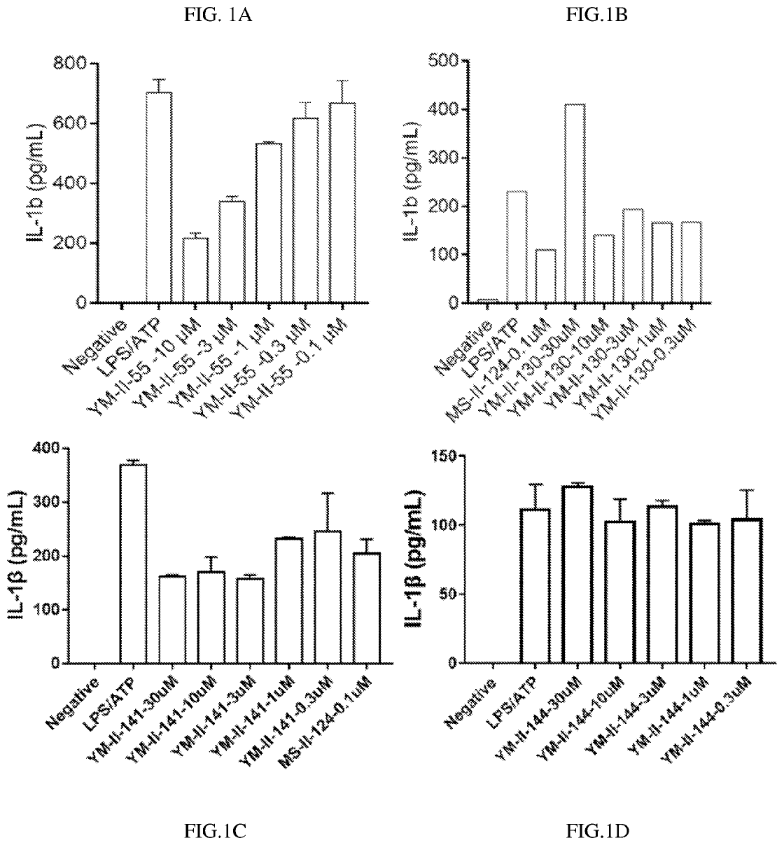

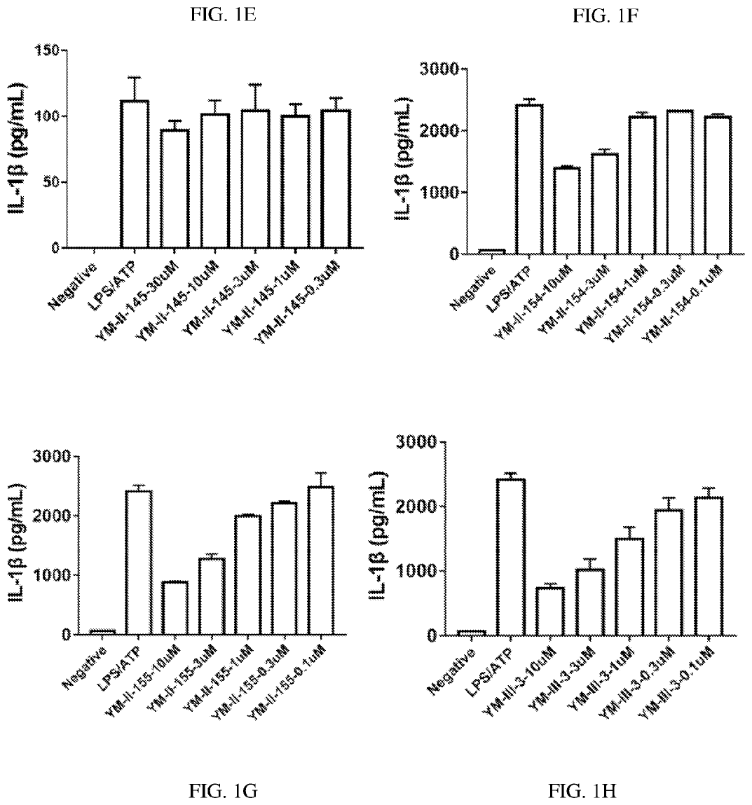

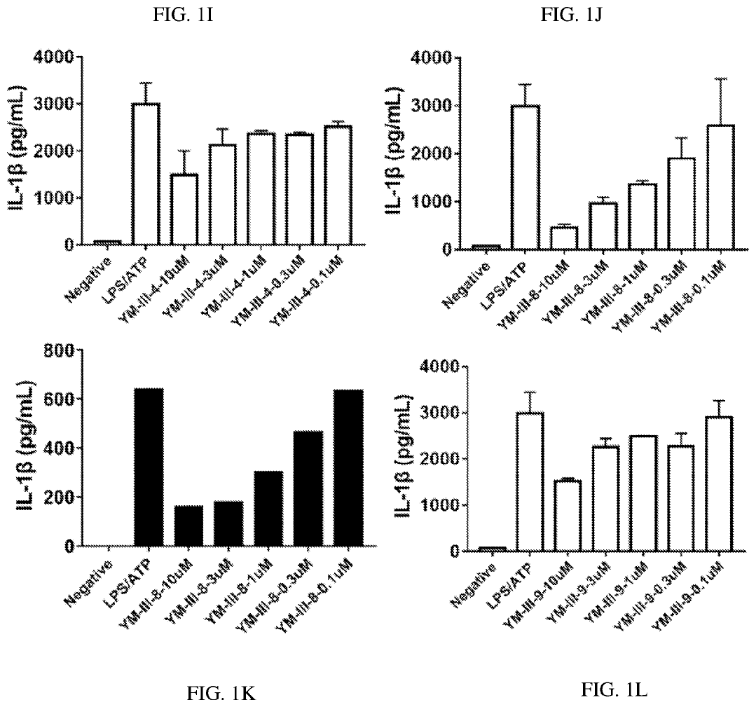

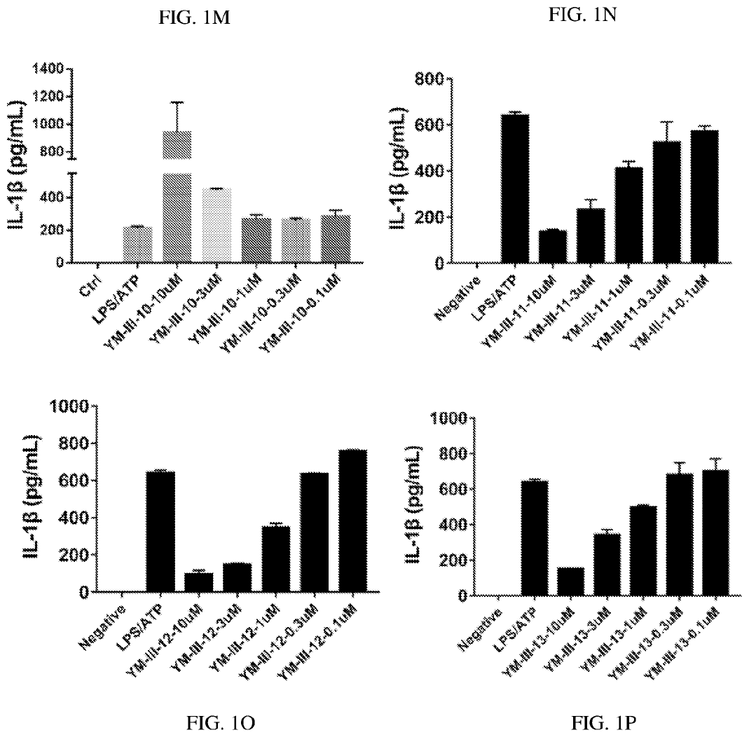

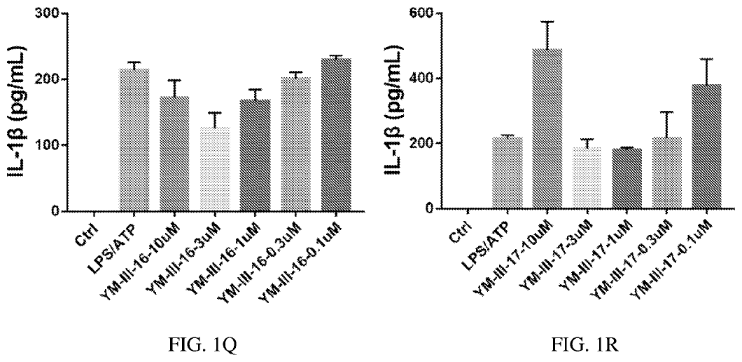

Cultured mouse macrophages were treated with LPS followed by ATP to induce the formation of the NLRP3 inflammasome and measure the release of mature IL-1B in the supernatant (FIGS. 1A-R). Treatment with NSIs started 30 minutes before ATP addition.

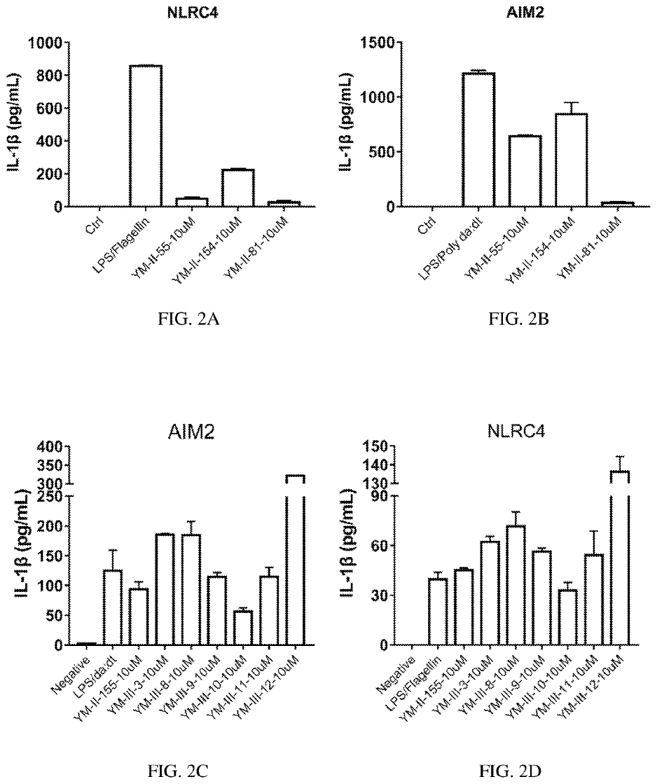

Cultured mouse macrophages were treated with poly(dA:dT) or flagellin followed by LPS and the release of mature IL-1β was measured in the supernatant (FIGS. 2A-D). Treatment with NSIs did not affect the release of IL-1β under these conditions (FIGS. 2A-D), indicating their selective inhibition on NLRP3 inflammasome activation.

### Binding to the Recombinant Mouse NLRP3 Protein

Monolith NT. Automated instrument was used to measure the Kd value. A range of concentrations of NSIs were incubated and His-NLRP3 pre-labeled with RED-tris-NTA dye kit (SKU: MO-L018) for 40 min in PBS-T assay buffer (1×PBS with 0.05% Tween® 20). The samples were loaded into NanoTemper® glass capillaries and series of ligand-induced changes in thermophoresis curves were gained using an Excitation power of 80% and a MST power of high. Kd values were calculated using the mass action equation with NanoTemper software from duplicate reads of an experiment.

Table 1 provides exemplary binding affinities of NSIs to human recombinant NLRP3 protein.

TABLE 1

| Compound | K_{D} (nM) |
|---|---|
| Formula III (YM-III-8) | 25 |
| Formula IV (YM-III-3) | 24 |
| Formula V (YM-III-12) | 980 |
| Formula VI (YM-III-11) | 3000 |
| Formula VII (YM-III-22) | 700 |
| Formula VIII (YM-III-23) | 480 |
| Formula IX (YM-III-18) | 860 |
| Formula X (YM-II-55) | 69 |

While the invention has been described in terms of its several exemplary embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. A compound or a pharmaceutically acceptable salt, solvate or hydrate thereof having the general chemical structure of Formula I: 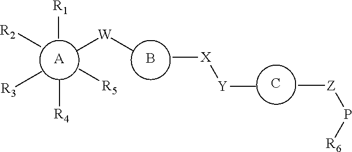
wherein
ring A is benzene, pyridine, pyrimidine, or 1,2-diazine;
R1 is unbranched, branched, saturated, unsaturated, cyclic or acyclic, substituted or unsubstituted C1-C8 alkyl or is unbranched, branched, saturated, unsaturated, cyclic or acyclic, substituted or unsubstituted C1-C8 alkoxyl, or is amino, nitro, OH, or halogen;
R4 is halogen, amino, nitro or cyano;
R2, R3, and R5 may be the same or different and are independently selected from
H,
C1-C8 unbranched, branched, saturated, unsaturated, cyclic or acyclic, substituted or unsubstituted alkyl,
C1-C8 unbranched, branched, saturated, unsaturated, cyclic or acyclic, substituted or unsubstituted alkoxyl,
C1-C8 unbranched, branched, saturated, unsaturated, cyclic or acyclic, substituted or unsubstituted alkylcarbonyl,
halogen,
hydroxyl,
amino,
nitro, and
cyano;
W is unbranched, branched, saturated, unsaturated, substituted or unsubstituted C1-C4 alkyl and may be present or absent;
ring B is 1,2,3-triazole, 1,2,4-triazole, 1,2-diazole, 1,3-diazole, pyrrole, 1,3-thiazole, or 1,3-oxazole;
X is unbranched, branched, saturated, unsaturated, substituted or unsubstituted C1-C4 alkyl and may be present or absent;
Y is O, NH, or S and may be present or absent;
ring C is benzene, pyridine, or another aromatic heterocycle;
Z is O, carbonyl, halogen, CF3, or C1-C8 unbranched, branched, saturated, unsaturated, cyclic or acyclic, substituted or unsubstituted alkyl;
P is O or S and may be present or absent; and
R6 is unbranched, branched, saturated, unsaturated, cyclic or acyclic, substituted or unsubstituted C1-C8 alkyl.

2. The compound of claim 1, wherein said compound has the general chemical structure of Formula II: 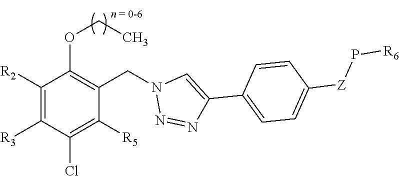
or
pharmaceutically acceptable salts, solvates, or hydrates thereof.

3. The compound of claim 1, wherein said compound is selected from the group consisting of 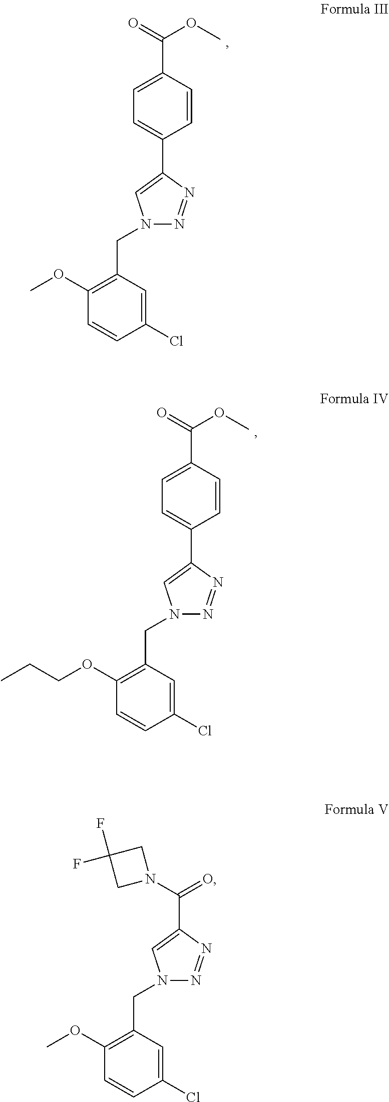 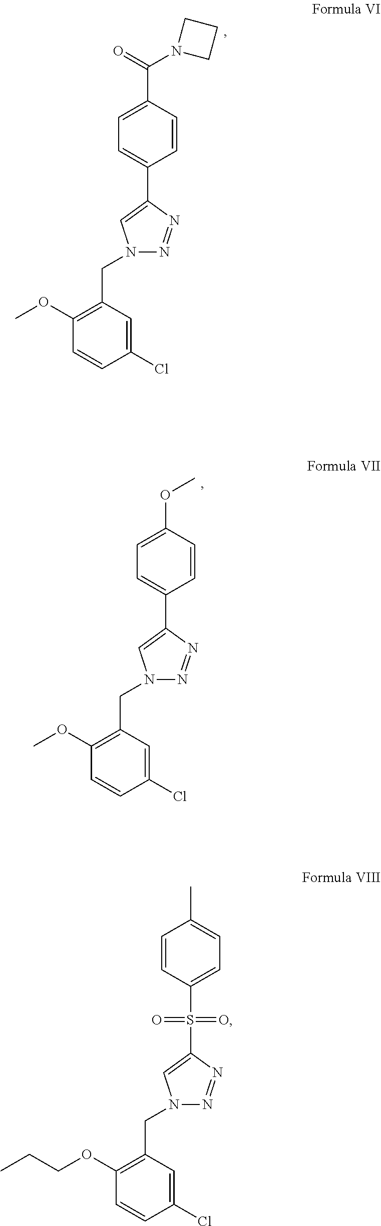 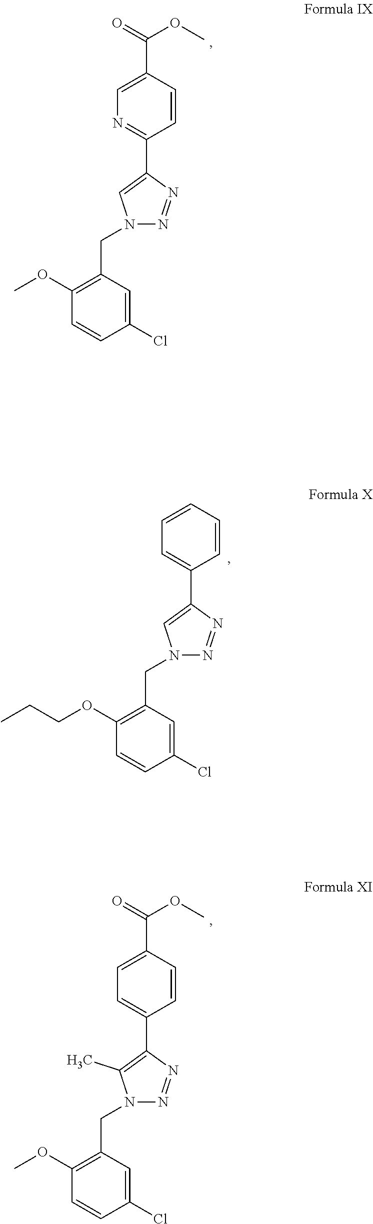 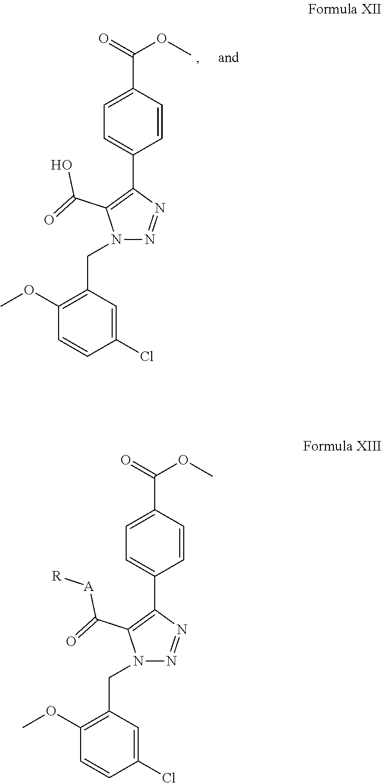
wherein
R is unbranched, branched, saturated, unsaturated, cyclic or acyclic, substituted or unsubstituted C1-C8 alkyl; and
A is O or NH.

4. A compound or a pharmaceutically acceptable salt, solvate or hydrate thereof having the general chemical structure of Formula XIV: 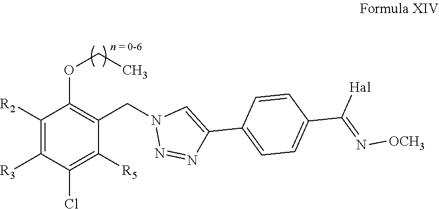
wherein
R2, R3, and R5 may be the same or different and are independently selected from
H,
C1-C8 unbranched, branched, saturated, unsaturated, cyclic or acyclic, substituted or unsubstituted alkyl,
C1-C8 unbranched, branched, saturated, unsaturated, cyclic or acyclic, substituted or unsubstituted alkoxyl,
C1-C8 unbranched, branched, saturated, unsaturated, cyclic or acyclic, substituted or unsubstituted alkylcarbonyl,
halogen selected from fluorine and chlorine,
hydroxyl,
amino,
nitro, and
cyano.

5. A compound or a pharmaceutically acceptable salt, solvate or hydrate thereof having the general chemical structure of Formula XV: 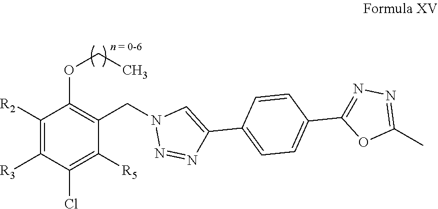
wherein
R2, R3, and R5 may be the same or different and are independently selected from
H,
C1-C8 unbranched, branched, saturated, unsaturated, cyclic or acyclic, substituted or unsubstituted alkyl,
C1-C8 unbranched, branched, saturated, unsaturated, cyclic or acyclic, substituted or unsubstituted alkoxyl,
C1-C8 unbranched, branched, saturated, unsaturated, cyclic or acyclic, substituted or unsubstituted alkylcarbonyl,
halogen,
hydroxyl,
amino,
nitro, and
cyano.

6. A compound or a pharmaceutically acceptable salt, solvate or hydrate thereof having the general chemical structure of Formula XVI: 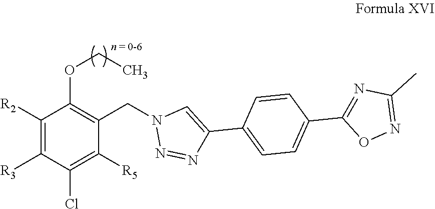
wherein
R2, R3, and R5 may be the same or different and are independently selected from
H,
C1-C8 unbranched, branched, saturated, unsaturated, cyclic or acyclic, substituted or unsubstituted alkyl,
C1-C8 unbranched, branched, saturated, unsaturated, cyclic or acyclic, substituted or unsubstituted alkoxyl,
C1-C8 unbranched, branched, saturated, unsaturated, cyclic or acyclic, substituted or unsubstituted alkylcarbonyl,
halogen,
hydroxyl,
amino,
nitro, and
cyano.

7. A composition comprising a compound of claim 1 and a pharmaceutically acceptable carrier.

8. A method of preventing or treating NRLP3 inflammasome-associated inflammation in a subject in need thereof, comprising a step of administering to said subject a therapeutically effective amount of a composition comprising at least one compound of claim 1 or a pharmaceutically acceptable salt, solvate or hydrate thereof.

9. The method of claim 8, wherein said subject suffers from Alzheimer's disease, Parkinson's disease, multiple sclerosis, traumatic brain injury, acute myocardial infarction, heart failure, arthritis, diabetes, gout, COVID-19, or an autoinflammatory condition.
